(19) United States
(12) Patent Application Publication
(10) Pub. No.: US 2003/0216677 A1
(43) Pub. Date: Nov. 20, 2003

(54) Biosensor for dialysis therapy

(76) Inventors: Li Pan, Tampa, FL (US); Ramesh Wariar, Tampa, FL (US); Leo Martis, Long Grove, IL (US); Cliff Holmes, Glenview, IL (US); Robert Childers, New Port Richey, FL (US); Shahid Din, Palm Harbor, FL (US)

Correspondence Address: BAXTER HEALTHCARE CORPORATION, RENAL DIVISION, 1 BAXTER PARKWAY, DF3-3E, DEERFIELD, IL, 60015, US

(21) Appl. No.: 10/147,568

(22) Filed: May 15, 2002

(51) Int. Cl. A61M 37/00; C02F 1/44; B01D 61/00

(52) U.S. Cl. 604/5.04; 604/5.01; 604/6.01; 210/739; 210/645; 210/252; 210/257.1

(57) ABSTRACT

A biosensor capable of monitoring a number of different constituents of a dialysate solution used during dialysis therapy is provided. The biosensor of the present invention includes an integrated array of reactive elements and sensing elements that can be hydraulically coupled to the dialysate solution. In this regard, the biosensor can be utilized to provide on-line monitoring of total solutes removed from a patient during dialysis therapy, infection levels and/or other desirable parameters such that an overall assessment of dialysis therapy can be readily evaluated.

### BACKGROUND OF THE INVENTION

The present invention relates generally to medical treatment. More specifically, the present invention relates to dialysis therapies.

Due to disease or insult or other causes, the renal system can fail. In renal failure of any cause, there are several physiological derangements. The balance of water, electrolytes (e.g., Na, K, Cl, Ca, P, Mg, SO₄) and the excretion of a daily metabolic load of fixed ions is no longer possible in renal failure. During renal failure, a variety of metabolic end products (e.g., urea, creatinine, uric acid, and the like) can accumulate in blood and tissues.

Dialysis processes have been devised for the separation of elements in a solution by diffusion as well as convection across a semi-permeable membrane. Principally, dialysis comprises two methods: hemodialysis; and peritoneal dialysis.

Hemodialysis treatment utilizes the patient's blood to remove waste, toxins, and excess water from the patient. The patient is connected to a hemodialysis machine and the patient's blood is pumped through the machine. Catheters or needles are inserted into the patient's veins and arteries to connect the blood flow to and from the hemodialysis machine. Waste, toxins, and excess water are removed from the patient's blood and the blood is infused back into the patient. Hemodialysis treatments can last several hours and are generally performed in a treatment center about three or four times per week.

Peritoneal dialysis utilizes a dialysis solution or dialysate, which is infused into a patient's peritoneal cavity through a catheter. The dialysate contacts the patient's peritoneal membrane in the peritoneal cavity. Waste, toxins, and excess water pass from the patient's bloodstream through the peritoneal membrane and into the dialysate. The transfer of waste, toxins, and water from the bloodstream into the dialysate occurs due to diffusion and osmosis. The spent dialysate is drained from the patient's peritoneal cavity to remove the waste, toxins, and water from the patient.

There are various types of peritoneal dialysis, including continuous ambulatory peritoneal dialysis, and automated peritoneal dialysis. In general, continuous ambulatory peritoneal dialysis is performed manually where fresh dialysate fluid is delivered via a catheter to the peritoneal cavity of the patient and remains there for a given dwell period subsequent to which the patient connects the catheter to a drain to allow spent dialysate fluid to drain from the peritoneal cavity. Automated peritoneal dialysis (APD) is similar to continuous ambulatory peritoneal dialysis in that the dialysis treatment includes a drain, fill, and dwell cycle. In contrast, APD is performed by a dialysis machine automatically where 3-4 cycles of peritoneal dialysis treatment is performed, typically overnight while the patient sleeps. Continuous flow peritoneal dialysis (CFPD) is a different modality of APD where multiple exchanges are replaced by continuously flowing dialysate into, through and out of the peritoneal cavity. Therefore, CFPD can significantly enhance solute clearance as a result of better mixing, the increased concentration gradient for diffusion and the increased peritoneal mass transfer coefficient.

In this regard, a dialysis machine can be fluidly connected to an implanted catheter. The dialysis machine can also be fluidly connected to a source of fresh dialysate, such as a bag of dialysate solution, and to a fluid drain. The dialysis machine can then pump the spent dialysate from the peritoneal cavity through the catheter to the drain. Thereafter, fresh dialysate from the dialysate source can be pumped through the catheter and into the patient's peritoneal cavity. The dialysis machine allows the dialysate to dwell within the cavity to transfer waste, toxins, and excess water from the patient's bloodstream to the dialysate solution. The dialysis system is computer controlled so that the dialysis treatment occurs automatically when the patient is connected to the dialysis system, for example, overnight.

Several drain, fill, and dwell cycles will occur during the treatment. Also, a last fill is typically used at the end of the automated dialysis treatment so that the patient can disconnect from the dialysis machine and continue daily functions while dialysate remains in the peritoneal cavity. Automated peritoneal dialysis frees the patient from manually performing the drain, dwell, and fill steps, and can improve the patient's dialysis treatment and quality of life.

In general, estimation of APD efficiency typically involves taking a dialysate sample from a bag of spent dialysate that has collected in the bag over a 24-hour period and a blood sample of the patient and having off-site laboratory analysis conducted in the samples. Based on the lab results and volume information from the APD machine, patient's clearance is manually calculated. However, the clearance calculation can be inaccurate due to errors in the manual calculation of clearance, in the lab analysis of the samples and/or other like conditions.

To this end, there exists a need to monitor dialysis treatment to ensure the proper administration and control of dialysis therapy. For example, monitoring of dialysis therapy may be needed to monitor the patient for infection that may occur during dialysis therapy, such as peritonitis, which can occur during peritoneal dialysis.

Further, there exists the need to ensure that toxins are effectively removed from the patient during dialysis. In general, use of time or duration of dialysis can be used as an indicator for an end point or completion of dialysis therapy. However, the use of time or duration as a dialysis end point indicator can be problematic, particularly as applied to patients whose response to dialysis does not follow predicted patterns or responses.

In this regard, a variety of different indices have been utilized to determine the effectiveness of dialysis treatment. In general, the indices can be derived from known transport mechanisms associated with dialysis therapy, e.g., the mass transfer of toxins from blood to dialysate fluid. The indices can be calculated based on measurable parameters of the dialysis system, such as the concentration of urea in the blood and dialysate. Known indices can include, for example Kt/V (where, in general, K=dialyzer clearance, t=time of dialysis and V=volume of distribution of urea), the time average concentration of blood urea, the protein catabolic rate and the urea reduction ratio. The measurable parameters are determined by utilizing conventional sensors and clinical laboratory analysis tools. The sensors are typically configured to detect or monitor a single component, such as urea concentration. However, the use of a single component or parameter as an indicator or marker for dialysis efficacy or adequacy is insufficient to monitor the patient. In this regard, it is generally accepted that there is no ideal single parameter to represent uremic toxins in blood and, thus, the removal thereof during dialysis therapy.

Accordingly, there exists a need to provide improved devices and methods for monitoring a number of treatment parameters such that dialysis therapy can be readily and effectively evaluated.

### SUMMARY OF THE INVENTION

The present invention provides improved devices and methods for monitoring and providing dialysis therapies. The present invention provides a biosensor that includes an integrated array of a number of different sensing mechanisms such that a variety of parameters can be evaluated at once and within a single sensing device during dialysis therapy. Multiple parameter monitoring can be conducted on-line and repeatedly over time. In this regard, an overall and in-depth assessment of dialysis therapy can be readily obtained.

For example, the biosensor of the present invention includes a number of different reactive elements, such as membranes that include enzymes which are reactive with solutes including toxins and other metabolic waste that are removed from a patient during dialysis therapy. The enzymes can break down the solutes, such as urea, into reaction by-products, such as ammonia and carbon dioxide. In an embodiment, the by-products can then be coupled to a number of sensing elements in hydraulic connection with the membranes. The sensing elements are capable of optically detecting the amount of by-products and/or the amount of additional other optically sensitive constituents of the dialysate via, for example, a colorimetric detection. The amount of optically sensitive constituents including the by-products which can be detected correlates to the amount of total solute(s) that was removed during therapy.

In this regard, day-to-day trends of total solute removals and/or clearance values can be determined and used as clinical markers, trend monitors, or indicators such that prescribed dialysis therapies can be effectively developed and performed. Thus, clinicians can adjust their prescriptions to patients based on daily dialysis marker trends such that dialysis effectiveness can be maximized.

Further, the biosensor of the present invention can be adapted to monitor a number of other parameters in addition to solute removal levels. For example, the present invention can monitor the pH and electrolytes (e.g., Na, K, Cl, Ca, P, Mg, the like or combinations thereof) of the dialysate, the presence of infection, response to antibiotic treatment of such infection within the patient during dialysis therapy, other desirable parameters and combinations thereof. With respect to monitoring infection, this can be performed by optically measuring the amount of, for example, total protein levels, white blood cell counts, bacteria, endotoxin levels, inflammatory mediators, like parameters and combinations thereof during dialysis therapy.

In an embodiment, the present invention provides an apparatus for on-line monitoring of multiple parameters associated with a dialysate solution discharged from a drain line of a dialysis system during dialysis therapy. The apparatus includes a housing enclosing a fluid circuit in hydraulic connection with the dialysate via the drain line containing a plurality of constituents. A number of reactive elements hydraulically connected to the fluid circuit within the housing are capable of reacting with at least a portion of the dialysate constituents to form a reaction product. Preferably, the reactive elements include enzymes, such as urease, creatinine deiminase, uricase, esterase, the like and combinations thereof. A number of sensing elements are hydraulically coupled to the fluid circuit within the housing. The sensing elements can be used to detect a number of different and suitable conditions, such as pH, electrolytes (e.g., sodium, potassium, calcium, magnesium, phosphate), infection, soluble mediators of infection, white blood cell count, total protein concentration, bacteria, endotoxin levels, other suitable conditions and combinations thereof. The sensing elements can detect optically the sensitive constituents of the dialysate solution including the reaction product associated with the constituents such that concentration of the constituents in the fluid is measurable. In an embodiment, the biosensor can be integrated within a pumping cassette used in a variety of different applications, such as during dialysis therapy.

Preferably, the constituents of the dialysate solution include one or more analytes representative of solutes removed from blood of a patient during dialysis therapy in addition to one or more constituents indicative of infection if present in the dialysate solution. In an embodiment, the constituents include urea, creatinine, uric acid, glucose, phosphates, bacteria, mediators of infection, endotoxin, white blood cells total protein like desirable constituents and combinations thereof.

The present invention also provides a method of on-line monitoring of multiple parameters associated with a dialysate solution used during dialysis therapy. The method includes the steps of providing a biosensor array hydraulically connected to the fluid channel wherein the biosensor array includes a housing that encloses a fluid circuit in fluid communication with a plurality of reactive elements and sensing elements; supplying the dialysate solution to the fluid circuit of the biosensor array wherein the dialysate solution includes a plurality of constituents reacting at least a portion of the constituents with the reactive elements to produce a reaction product associated with the constituents; optically measuring the reaction product associated with the constituents via the sensing elements; and determining an amount of the constituents in the dialysate solution.

Preferably, at least a portion of the constituents of the dialysate solution enzymatically reacts with an enzyme as discussed above. In this regard, the present invention can provide on-line monitoring levels of solutes removed from the patient dialysis therapy, the presence of infection and/or other desirably monitored parameters of dialysate used during dialysis therapy.

For parameters, such as urea and creatinine, a hydrophobic membrane impregnated with an indicator is preferably used to measure NH₃ and CO₂ as discussed in detail below. Before each successive measurement, the membrane is rinsed completely. This facilitates the ability of the biosensor to conduct multiple online measurements. With this ability, clearance calculations can be effectively made based on the data generated from the multiple and successive measurements. In this regard, the biosensor can be adapted to provide on-line measurements of a number of parameters, for example, the long dwell dialysate to estimate plasma levels, the waste dialysate after each therapy to evaluate total solutes removed and/or the like.

In another embodiment, a method for providing dialysis therapy is provided. The method includes the steps of removing one or more toxins from the patient with the dialysate solution; supplying the dialysate solution to a biosensor array within a housing that encloses a fluid circuit in fluid communication with a plurality of reactive elements and a plurality of sensing elements wherein the dialysate solution includes a plurality of constituents including solutes removed from the patient; reacting at least a portion of the constituents of the dialysate solution with the reactive elements to produce a reaction product associated with the constituents optically sensing at least a portion of the constituents including the reaction product with the sensing elements; and determining an amount of the in the dialysate solution.

Preferably, the method provides on-line monitoring of the constituents of the dialysate solution including one or more analytes representative of solutes removed during dialysis therapy and one or more constituents indicative of infection, the response to infection therapy, such as infection due to peritonitis, in the dialysate solution and/or the response to infection therapy.

An advantage of the present invention is to provide an improved device and method for monitoring dialysis therapy including peritoneal dialysis.

Moreover, an advantage of the present invention is to provide an improved device and method for monitoring a number of different parameters during dialysis therapy, including, for example, the removal of solutes, infection and response to antibiotic therapy in a patient such that an overall evaluation of dialysis therapy can be readily assessed.

Another advantage of the present invention is to provide an improved device and method to determine clearance in dialysis based, in part, on measurable amounts of solutes removed during dialysis therapy.

Still another advantage of the present invention is to provide an on-site tool to determine a patient's peritoneum solute transport characteristics during peritoneal dialysis including, for example, a mass-transition area coefficient (MTAC), a peritoneal equilibrium test (PET) which is required for a patient's prescriptions during APD and/or the like.

Yet another advantage of the present invention is to provide an improved biosensor which includes an integrated array of reactive elements and sensing elements in hydraulic communication with a dialysate solution to optically detect a measurable amount of constituents in the dialysate solution including solutes removed from the patient and infection and response to treatment for infection.

Still yet another advantage of the present invention is to provide an improved biosensor that can detect both the removal of solutes from a patient and the presence of infection in the patient during dialysis therapy.

A further advantage of the present invention is to provide an improved apparatus and method for conducting multiple parameter and on-line sensing of solute removal levels, clearance levels, infection levels and electrolyte balances (e.g., sodium, potassium, calcium, magnesium, bicarbonate, other suitable electrolytes or combinations thereof) during dialysis therapy.

A still further advantage of the present invention is to provide an improved biosensor that employs colorimetric detection to measure and monitor an amount of constituents of a dialysate, such as solutes removed during dialysis therapy and infection in the dialysate.

Additional features and advantages of the present invention will be described in and apparent from the detailed description of the presently preferred embodiments and the figures.

### BRIEF DESCRIPTION OF THE FIGURES

FIG. 1 illustrates schematically a biosensor of an embodiment of the present invention.

FIG. 2A illustrates schematically a biosensor and a dialysis system in hydraulic connection and wireless communication according to an embodiment of the present invention.

FIG. 2B illustrates schematically a biosensor and a dialysis system in hydraulic connection and wireless communication according to an embodiment of the present invention.

FIG. 2C illustrates schematically a biosensor and a dialysis system in hydraulic connection and wireless communication according to an embodiment of the present invention.

FIG. 3A illustrates an embodiment of the present invention showing a top view of the biosensor integrated within a pumping mechanism.

FIG. 3B illustrates an embodiment of the present invention showing a bottom view of the biosensor integrated within a pumping mechanism of FIG. 3A.

### DETAILED DESCRIPTION OF THE INVENTION

The present invention provides devices and methods for monitoring and evaluating dialysis therapy. More specifically, the present invention provides a biosensor array that is capable of monitoring a number of different parameters at once and repeatedly over time during dialysis therapy such that an overall assessment of dialysis therapy can be readily and easily provided.

It should be appreciated that the present invention can be used in a variety of different dialysis therapies to treat kidney failure. Dialysis therapy as the term or like terms are used throughout the text is meant to include and encompass any and all forms of therapies that utilize the patient's blood to remove waste, toxins and excess water from the patient. Such therapies include hemodialysis, hemofiltration, hemodiafiltration and peritoneal dialysis including automated peritoneal dialysis continuous ambulatory peritoneal dialysis and continuous flow peritoneal dialysis. Such therapies can also include, where applicable, both intermittent therapies and continuous therapies used for continuous renal replacement therapy (CRRT). Examples of continuous therapies used in CRRT include slow continuous ultrafiltration (SCUF), continuous venovenous hemofiltration (CVVH), continuous venovenous hemodialysis (CVVHD), continuous venovenous hemodiafiltration (CVVHDF), continuous arteriovenous hemofiltration (CAVH), continuous arteriovenous hemodialysis (CAVHD), continuous arteriovenous hemodiafiltration (CAVHDF), continuous ultrafiltration periodic intermittent hemodialysis or the like.

Further, although the present invention, in an embodiment, can be utilized in methods providing a dialysis therapy for patients having chronic kidney failure or disease, it should be appreciated that the present invention can be used for acute dialysis needs, for example, in an emergency room setting. Lastly, as one of skill in the art appreciates, various forms of dialysis therapy, such as hemofiltration, hemodialysis, hemodiafiltration and peritoneal dialysis may be used in the in center, self/limited care as well as the home settings.

In an embodiment, the biosensor of the present invention can be effectively utilized to perform multiple parameter on-line sensing of total analyte or solute removals. The present invention, thus, has the capability to provide day-to-day measurements of solute removal levels from a patient during dialysis. In this regard, the solute removal measurements in addition to concentrations of the solutes measured at certain time intervals prior to removal can be utilized to determine and evaluate indices of dialysis adequacy on a day-to-day basis. The solutes can include any typical and desirably monitored solute removed from the patient during dialysis therapy. Solute examples include urea, creatinine, uric acid, glucose, phosphates and/or the like.

The day-to-day trend of total solute removals and/or clearances can be effectively utilized as clinical markers to evaluate the adequacy of prescribed dialysis therapies. Based on the measurable daily dialysis biomarker trends, clinicians would then have the ability to adjust their prescriptions to patients such that the effectiveness of dialysis therapy can be maximized.

In addition to monitoring solute removal levels, the biosensor of the present invention, in an embodiment, also has the capability to monitor the presence and severity of infection during a dialysis treatment. The markers of infection pass from the patient into the dialysis solution. If detected in the dialysate, treatment of the infection by, for example, antibiotics or other suitable medicinals, can be initiated. The progress of infection treatment can then be monitored using the same sensor.

This is particularly important as applied during peritoneal dialysis where peritonitis is known to occur. In this regard, the biosensor array, in an embodiment, can readily provide an overall assessment based on, for example, how effectively solutes are removed from the patient and/or how much, if any, infection exists in the patient during dialysis therapy.

The biosensor of the present invention, in general, includes an integrated array of a variety of different and suitable configurations to provide effective monitoring of a number of parameters during dialysis therapy. In an embodiment, the biosensor includes a housing that encloses a fluid circuit which is hydraulically coupled to the dialysate used during therapy. The fluid circuit includes a number of reactive elements and sensing elements in hydraulic connection with the fluid. The reactive elements are capable of reacting with at least a portion of the constituents of the fluid that are reactive. This can yield a reaction product associated with the constituents. The reactive elements can include a variety of different and suitable materials including enzymes, chemical agents, and the like that are reactive with at least a portion of the constituents to produce an optically sensitive product.

The sensing elements are capable of optically measuring the constituents of the fluid including the reaction product associated with the constituents such that an amount of the constituents in the fluid can be determined. The reactive elements, such as enzymes or indicators, and the sensing elements can be supported by or impregnated onto a carrier member or media. The carrier media can support the reaction and sensing elements separately and/or together. The carrier media can include any suitable material, such as a membrane, a substrate composed of a fibrous material including paper, like support materials and combinations thereof. It should be appreciated that the sensing elements and the reactive elements can include a variety of suitable materials, examples of which are detailed below.

In an embodiment, the biosensor of the present invention can include a variety of different constituents such that it can effectively perform multiple parameter on-line sensing of a dialysate solution during dialysis therapy, including peritoneal dialysis. The parameters include, for example, total analyte or solute removals, infectious agents, inflammatory mediators of infection, pH, electrolytes, like parameters and combinations thereof. In an embodiment, the biosensor array includes a housing that encloses an enzyme membrane and a sensing membrane in hydraulic connection with the enzyme membrane layer.

The enzyme membrane layer can include a number of different enzyme membranes which are reactive with a number of different constituents of the dialysate such that a number of different optically sensitive reaction products can be produced. The sensing membrane layer can include a variety of different sensing membranes that are optically responsive to the reaction products produced from the enzyme membrane layer in addition to additional other optically sensitive constituents of the dialysate. By optically responsive, it should be appreciated that the present invention can include a variety of suitable optical detection techniques that can be utilized. These can include, for example, light scattering techniques, fluorescence techniques, colorimetery, the like and combinations thereof. In this regard, the biosensor array can measure the amount of a variety of different optically sensitive constituents of the dialysate, such as solutes that have been removed during dialysis therapy.

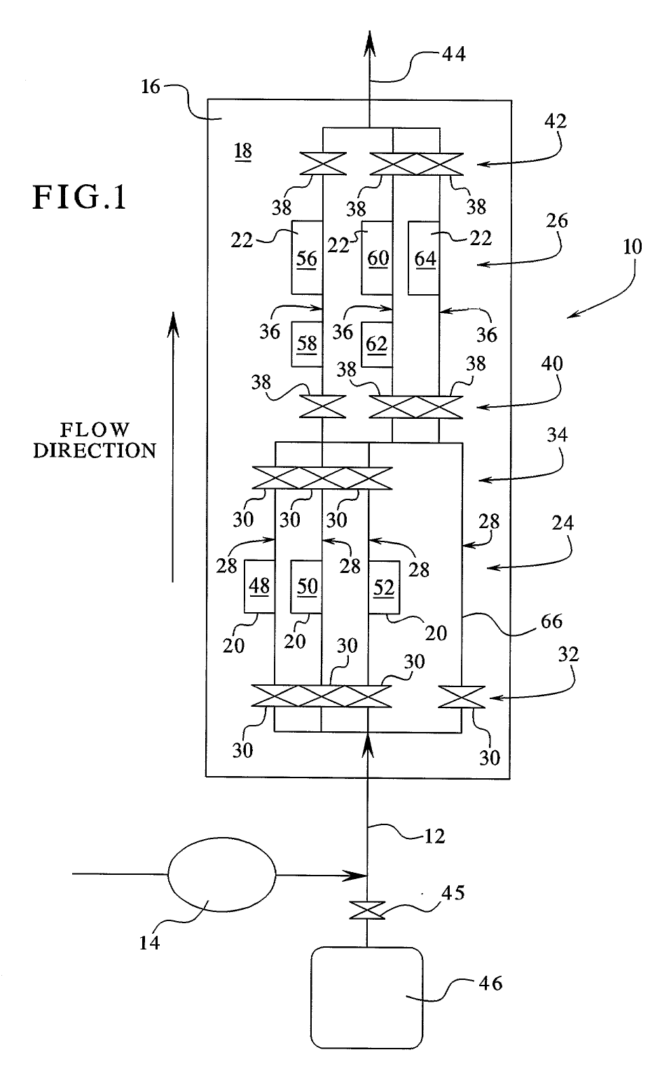

In an embodiment, the biosensor 10 is in hydraulic connection with a fluid channel 12 as illustrated in FIG. 1. The fluid channel 12 can supply any suitable fluid or solution, such as a dialysate solution, to the biosensor array. The biosensor array 10 can measure a variety of constituents of the dialysate. In an embodiment, the dialysate can be delivered to the biosensor array via the fluid channel by a suitable pumping mechanism 14.

The biosensor can include a variety of suitable configurations. In an embodiment, the biosensor array 10 includes a mini-cassette 16 configuration. In this regard, the housing 18 of the mini-cassette can include a variety of suitable dimensions and shapes. In an embodiment, the housing 18 has an equal width and length of approximately 1.5 inches. It should be appreciated that the housing 18 can be made from a variety of different and suitable materials including, for example, rigid plastic materials, flexible plastic materials or other like materials that can be utilized to protect the constituents of the biosensor.

In an embodiment, the biosensor 10 of the present invention includes a number of enzyme membranes 20 and sensing membranes 22 in fluid connection with the enzyme membranes as illustrated in FIG. 1. The enzyme membranes and sensing membranes are located within an enzyme membrane layer 24 and a sensing membrane layer 26, respectively, as previously discussed.

In an embodiment, the enzyme membranes 20 are connected to the fluid channel 12 through a series of fluid channels 28 within the housing 18 of the biosensor as further illustrated in FIG. 1. It should be appreciated that the enzyme membranes 20 can be connected to the fluid channel 12 in a variety of different and suitable ways. In an embodiment, each membrane is separately connected to the fluid channel via a fluid channel enclosed within the housing of the biosensor. As further illustrated in FIG. 1, the fluid channels 28 connected to the enzyme membrane 20 can each have control valves 30 positioned at an inlet side 32 and outlet side 34 of the fluid channels 28 to regulate the flow rate of the fluid supplied from the fluid channel 12.

In an embodiment, the fluid is then supplied to the sensing membrane layer 26 via a series of fluid channels 36 hydraulically connected to the enzyme membrane layer 24 as illustrated in FIG. 1. In an embodiment, the fluid channels 36 that connect the sensing membranes 26 to the enzyme membranes 24 can include control valves 38 at an inlet side 40 and an outlet side 42 of the fluid channels 36 with respect to the sensing membranes 26. As further illustrated in FIG. 1, the fluid can exit the biosensor 10 through a single exit fluid channel 44 after it passes through the sensing membrane 26.

It should be appreciated that the biosensor array of the present invention can include a variety of fluid circuit configurations containing a number of different constituents, e.g., fluid channels, enzyme membranes, sensing membranes, control valves and the like, such that the amount of constituents in solution which are desired to be measured can be effectively monitored. For example, the biosensor can be adapted such that the reactive elements and the sensing elements can be readily removed and replaced during therapy. This allows the user with the ability to adjust and control the number of parameters to be monitored. In this regard, the biosensor can be modified to monitor one or more different parameters at any time during therapy.

In an embodiment, about two to about three milliliters of fluid is necessary for each measurement. In an embodiment, the sensing membranes include a chamber for detecting the optically-sensitive constituents, such as the analytes in solution, which is about 0.5 ml in volume.

It should be appreciated that the enzyme layer 24 can include a variety of configurations. In an embodiment, the enzyme layer 24 includes three separate enzyme membranes as illustrated in FIG. 1. The biosensor can also include any suitable number of additional reactive membranes, including enzyme membranes that can react with a number of other constituents of the fluid to yield an optically-sensitive reaction product of the constituents.

In an embodiment, the fluid can be delivered via a valve 45 and the pump mechanism 14, such as a mini-pump or the like, from a solution bag or the drain bag 46 that contains a number of different constituents which are reactive with the enzyme membrane of the biosensor array. In a preferred embodiment, the enzyme membranes are reactive with solutes or analytes removed during dialysis therapy, constituents representative of infection in the dialysate, like desired constituents and combinations thereof. In this regard, the dialysate solution contains a number of solutes and/or other constituents, such as ultrafiltrates, that have passed from the blood of a patient to the dialysate solution during dialysis therapy. The solutes of the dialysate solution can include toxic end products associated with nitrogen metabolism, such as urea, creatinine, uric acid, glucose and the like which can accumulate in blood and tissues during renal failure, other metabolic wastes, such as phosphates, and/or the like.

As the dialysate solution is supplied to the biosensor, at least a portion of the solutes contained within the dialysate solution are reactive with enzymes supported in the enzyme membranes. The enzymes can include a variety of different enzymatic materials, such as urease, creatinine deiminase, uricase, or the like. Each enzyme is reactive with a specific type of analyte such that certain reaction products are produced, including, for example, ammonia, ammonium, carbon dioxide, hydrogen ions or the like.

In an embodiment, each enzyme membrane of the biosensor is reactive with a different type of analyte such that certain reaction products specific to these analytes are produced. As illustrated in FIG. 1, the enzyme layer of the biosensor includes enzyme membranes containing urease, creatinine deiminase and uricase. In this regard, each of the enzymes are reactive with a specific analyte, including, for example, urea, creatinine and uric acid.

For example, the enzyme membrane containing urease 48 can enzymatically convert urea and water into ammonia and carbon dioxide; the enzyme membrane containing creatinine deiminase 50 can enzymatically convert creatinine into ammonia, ammonium and N-methylhydantoin; and the enzyme membrane containing uricase 52 can enzymatically convert uric acid, water and oxygen into carbon dioxide, allantoin and hydrogen peroxide.

In an embodiment, the reaction products are then supplied to the membrane sensing layer 26 via the fluid circuit of the biosensor as illustrated in FIG. 1. In this regard, the sensing membrane layer 26 provides a series of sensing membranes which are optically responsive or sensitive to at least a portion of the constituents of the fluid including the reaction products of the constituents, such as ammonia, carbon dioxide, hydrogen ion, electrolytes (e.g., sodium, magnesium, calcium, other suitable electrolytes or combinations thereof), the like or combinations thereof in addition to other optically sensitive constituents contained in the fluid.

In an embodiment, the reaction products from the urease enzyme membrane are supplied to a sensing membrane 56 that is calorimetrically sensitive to ammonia and ammonium as illustrated in FIG. 1. In an embodiment, the urease membrane 48 reaction products are supplied to the sensing membrane 56 via a pH conditioner 58, such as magnesium oxide or the like, in order to increase the pH of the solution to approximately pH 10. In an embodiment, the reaction products associated with the creatinine deiminase enzyme membrane 50 are supplied to the ammonia and ammonium sensing membrane 56 via the pH conditioner 58 similar to the reaction products of the urease enzyme membrane 48.

In an embodiment, the reaction products of the uricase enzyme membrane 52 are supplied to a sensing membrane 60 which is calorimetrically sensitive to changes in the level of carbon dioxide. In an embodiment, the uricase reaction products are subjected to a pH conditioner 62, such as a solid phase acid of molybdenum trioxide, prior to carbon dioxide detection at the sensing membrane.

In an embodiment, a portion of the fluid via the fluid channel 66 can be supplied to the sensing membrane 64 thereby by-passing the reactive membrane layer. This can be utilized for pH detection, detection of infection or other like single stage monitoring parameters as discussed below.

In an embodiment, a fluid other than the dialysate can be fed through this fluid channel or other desirable fluid channels of the biosensor to monitor one or more parameters. For example, a calibration fluid derived from a fresh dialysate source can be fed through the biosensor in addition to and/or separately from the waste dialysate. In this regard, parameters of the dialysate, such as pH or the like, can be monitored before and after removal of solutes, excess water and/or other metabolic wastes from the patient. The monitoring of the fresh dialysate source can also be utilized for calibration purposes.

It should be appreciated that the membranes (e.g., enzyme membranes, sensing membranes) can be prepared in any suitable manner. Any suitable membrane can be used such that an enzyme or enzymes or other suitable chemically and/or biologically reactive agent can be properly adhered to and/or impregnated into the membrane in order to react with the constituents of the fluid, such as urea, creatinine or other like metabolic waste from the patient and retained in the dialysate.

As previously discussed, the biosensor array of the present invention can be effectively utilized to monitor and/or detect a number of analytes representative of solutes removed during dialysis therapy. In an embodiment, the calorimetric sensing membranes are coupled to opto-electronic circuits (not shown) which can be located outside of the mini-cassette or housing of the biosensor array. In this regard, the optoelectronic circuits can be utilized to convert the calorimetric responses of the sensing membranes into concentration values associated with the measured analytes.

In an embodiment, the concentration values can be further utilized to determine clearance values associated with dialysis therapy. The clearance can be calculated in any suitable manner, such as by utilizing any suitable clearance index or modification thereof, such as Kt/V in hemodialysis. With the capability to measure a number of different analytes, the biosensor array of the present invention can be effectively utilized to conduct multiple parameter on-line sensing of total solute removals and/or clearances. Clearance calculations in accordance with an embodiment of the present invention are discussed in detail below.

In addition to monitoring solute removal levels during dialysis therapy, the biosensor of the present invention can also monitor a number of other parameters, examples of which include the pH of the dialysate (fresh and/or waste), markers of infection in the dialysate, like parameters desired to be monitored and combinations thereof.

The capability to monitor for infection is important during dialysis therapy, particularly during peritoneal dialysis where peritonitis is known to occur. It should be appreciated that the present invention can include any variety and suitable type of sensing mechanism which is capable of detecting infection. In an embodiment, the sensing mechanism is capable of monitoring infection markers by detecting the concentration of protein in dialysate. In an embodiment, the sensing mechanism is capable of monitoring infection by detecting a white blood cell count in the fluid. In an embodiment, the sensing mechanism is capable of monitoring infection by detecting soluble mediators of inflammation, which are produced or levels of which change during periods of infection. Soluble mediators of infection include cytokines such as IL-6, IL-1, and TNFα, chemokines, such as IL-8, MCP-1, MIP-1 and RANTES. Other soluble mediators of infection include arachadonic acid pathways metabolites, including thromboxane, leukotriene and cyclooxygenase products. Other soluble mediators of infection are complement cascade products such as C3a, C5a, C3b, C3bi, and the Membrane attack complex, C5b-9. Other soluble mediators of infection include adhesion molecules such as ICAM-1, VCAM-1 and the selectin molecules. In an embodiment, the sensing mechanism is capable of monitoring infection by detecting any suitable combination of parameters typically associated with the detection of infection, such as, protein levels, white blood cell count, endotoxins, bacteria, soluble mediators of infection, and the like.

In a preferred embodiment, trend monitoring for detection of infection is based on the optical detection of white blood cell counts on the patient. It is believed that this can be used as an effective marker for the presence of infection at an early stage such that the infection can be effectively and reversibly treated. During treatment, white blood cell counts can continue to be monitored to evaluate the effectiveness of the antibiotics and/or other like medicinals or drug administered to the patient during treatment of, for example, peritonitis.

The infection sensing mechanism, in an embodiment, can be designed for multiple use or single use monitoring. As applied to multiple use monitoring, the infection sensing mechanism includes, in an embodiment, one or more infection sensing membranes capable of detecting total proteins, white blood cell counts, bacteria, bacterial cell wall components including, for example, lipopolysaccharide (endotoxin), lipid A, peptidoglycan, muramyl peptides, β glycans or like constituents in the fluid, such as the dialysate that may be potentially associated with infection. In this regard, the infection sensing mechanism can be used a repeated or multiple number of times without having to replace it with another sensing mechanism.

In an embodiment, the infection sensing membrane contains one or more agents which are sensitive to the amount and types of fluid constituents typically associated with infection (e.g., proteins, white blood cells or the like) such that the constituents can be optically detected. In an embodiment, the agent is colorimetrically responsive to the concentration of constituents associated with infection in the dialysate. It should be appreciated that any variety of suitable opto-electronics or other like devices can be coupled to the infection sensing membranes to convert the calorimetric response of the membrane into a concentration value associated with the respective component, such as the total amount of protein.

As applied to single use, the infection sensing mechanism must necessarily be replaced after each use. It should be appreciated that the infection sensing mechanism can include a variety of suitable constituents to monitor infection in a single use application. In an embodiment, the infection sensing mechanism includes one or more substrates made from a fibrous material including paper, such as commercially available test strips which are calorimetrically responsive to one or more constituents (e.g., white blood cell counts) in the dialysate typically used as indicators for infection. Examples of such commercially available test strip products include SERIM PERISCREEN TESTS STRIPS which utilize esterase to promote the calorimetric detection of white blood cells in solution.

It should be appreciated that single use monitoring can be conducted for any suitable component of the dialysate in addition to infection levels. For example, any suitable solute can be measured in a single use manner. In an embodiment, glucose, phosphates and/or other like solutes can be measured in that way. With respect to glucose and commercially dry chemical strips or substrates that include an active agent which is calorimetrically responsive to glucose or phosphate levels can be used. Glucose and phosphate chemical strips are well known in the art.

The biosensor of the present invention can be adapted in a variety of suitable ways to monitor infection levels in the dialysate. Similar to FIG. 1, the biosensor can include two separate layers in hydraulic connection to monitor infection like the amount of analytes in the dialysate solution. In this regard, the fluid circuit of FIG. 1 can be expanded to include an additional number of separate fluid channels to monitor infection by colormetrically measuring, for example, the white blood cell count, the concentration of total proteins, bacteria and endotoxins in the fluid. The additional fluid channels may include a separate reactive layer to produce optically sensitive byproducts of the infection and a sensing layer which can optically sense the by-products in addition to other optically sensitive constituents of the fluid indicative. As well, the infection level can be optically sensed using a single component where the enzyme and sense layers are combined. This can be performed by utilizing, for example, commercially available test strips, such as the SERIM PERISCREEN TEST STRIPS.

It should be appreciated that the term “membrane” or other like terms as used herein means any suitable material that can effectively act as a carrier for agents that are sensitive, reactive, responsive or the like to changes in the surrounding environment in contact with the membrane, such as changes in a fluid in contact with the membrane. For example, the term “enzyme membrane” or other like terms as used herein means a membrane that contains one or more enzymes which are capable of reacting with certain constituents of a fluid in contact with the membrane, such as constituents of a dialysate which were removed from a patient's blood during dialysis therapy (e.g., urea). The term “sensing membrane” or other like terms as used herein means a membrane that contains one or more suitable agents which are, for example, calorimetrically responsive to certain constituents in contact with the membrane, such as constituents (e.g., carbon dioxide) produced from the enzymatic reaction of other constituents (e.g., urea) in the enzyme membranes. The term “infection sensing membrane” or other like terms means a membrane that contains one or more suitable agents which are, for example, calorimetrically responsive to constituents of a fluid in contact with the infection sensing membrane which are typically associated with infection in the fluid. It should be appreciated that the membranes of the present invention can be prepared in any suitable manner. For example, the membranes can be prepared as disclosed in U.S. patent application Ser. No. 10/024,670, and entitled “Hydrophobic Ammonia Sensing Membrane”, the disclosure of which is incorporated herein by reference.

In an embodiment, the present invention provides methods for monitoring a number of parameters specific to dialysis therapy, such as the detection of solutes removed from the patient and/or the degree of infection in the patient during dialysis therapy. In this regard, the biosensor of the present invention can be coupled to a dialysis system in any suitable manner such that the dialysate or other suitable fluid can be effectively monitored. It should be appreciated that the biosensor can be utilized with any suitable dialysis system, including any suitable number and type of constituents. For example, the biosensor can be utilized with hemodialysis systems and peritoneal dialysis systems including cyclers used in automated peritoneal dialysis, such as Home Choice™ which is commercially available from the BAXTER HEALTHCARE CORPORATION.

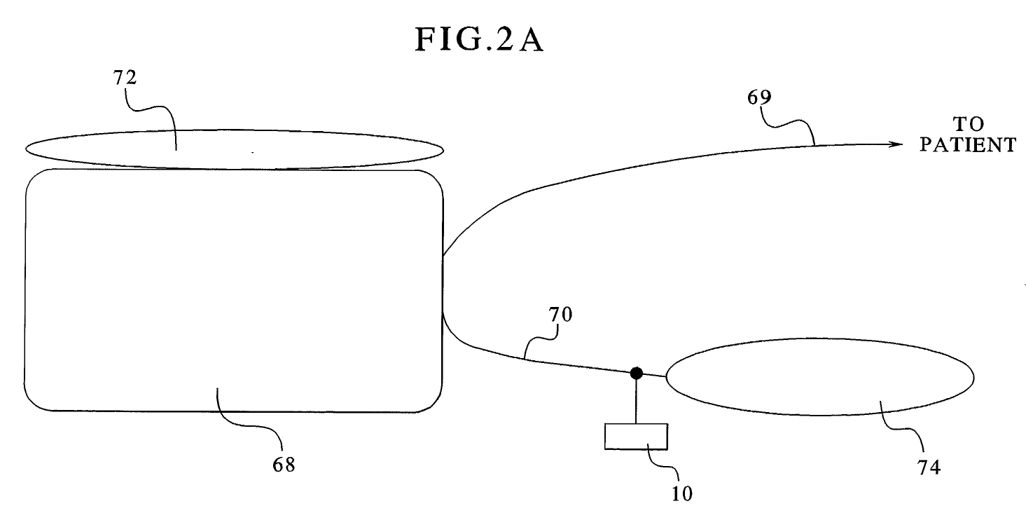

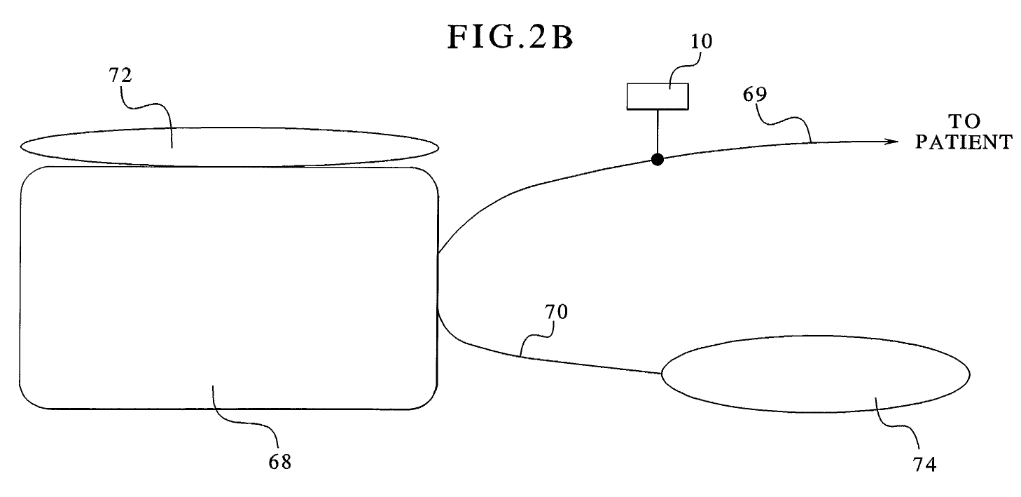

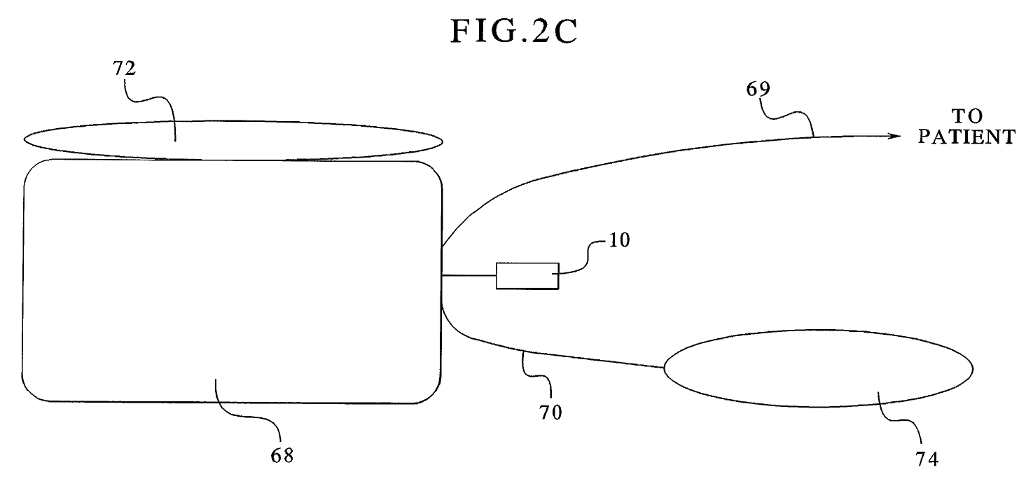

In an embodiment, the biosensor array 10 can be hydraulically connected to a suitable dialysis system 68, via a fluid channel 70 as illustrated in FIG. 2A. During dialysis therapy, a dialysate solution is supplied to the dialysis system via a solution bag 72. The dialysate solution is then utilized to remove excess water and solutes including toxins and other metabolic waste from the blood of a patient. The biosensor array can then be supplied with a dialysate solution drained from the patient that contains a number of constituents representative of the solutes removed during dialysis therapy in addition to a fresh dialysate solution for calibration purposes. The waste dialysate and fresh solution can both be fed to a drain bag 74 and the biosensor array 10. The fresh dialysate can be separately fed to the biosensor (not shown). The biosensor array 10 can also be coupled to the patient line 69 (FIG. 2B) or the dialysis system 68 (FIG. 2C) instead of the drain line 70. It should be appreciated that a biosensor array can be separately connected to the drain line in addition to the patient line and the dialysis machine or any suitable other combination of the drain line, the patient line and the dialysis machine.

In this regard, the biosensor can simultaneously monitor both fresh and waste dialysate. The fresh dialysate may be monitored for a number of different conditions. For example, a pH sensor can be utilized to monitor the fresh dialysate. This can be particularly important when two separate fluid constituents are mixed prior to therapy, where one component has a low pH and the other component has a high pH, such as a lactate-based solution and a bicarbonate-based solution, respectively. If by error, only one component is infused during therapy, the pH sensor can be used to detect the incorrect pH before the solution is delivered to the patient. The monitoring of the fresh dialysate can also be used to establish a baseline level in the dialysate from which the detectable amount of constituents in the waste dialysate can be compared for calibration purposes.

In an embodiment, the biosensor array can include a stand-alone device that is hydraulically connected to the dialysis system 68 via the fluid channel 70 as shown in FIG. 2A. It should be appreciated that the present invention is not limited to a standalone configuration. In this regard, the biosensor and dialysis system can be coupled in any suitable way. For example, the biosensor can be an integral component of the dialysis machine, pumping cassette or the like.

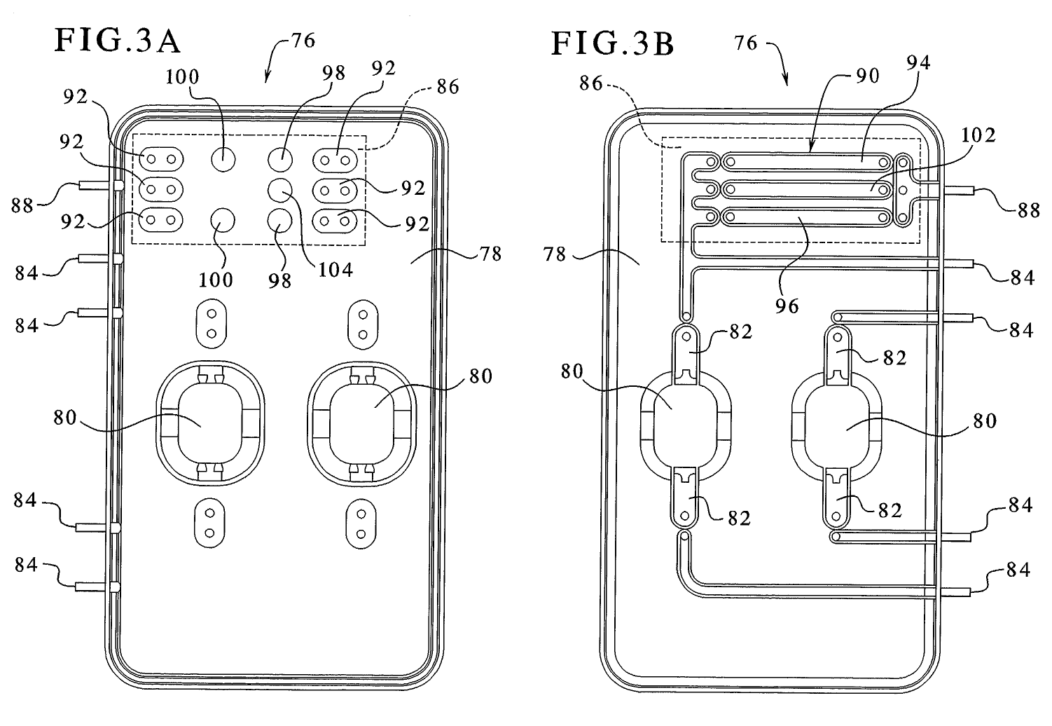

In an embodiment, the biosensor of the present invention can be integrated within a pumping cassette 76 used during dialysis therapy as shown in FIGS. 3A and 3B. In general, the pumping cassette 76 includes a housing 78 that encloses a number of fluid lines coupled to one or more operational constituents, such as a pumping mechanism that is capable of causing dialysis solution to flow to and from the patient (not shown) during dialysis therapy. The pumping cassette 76 can include any variety of suitable pumping cassettes and modifications thereof, such as a cycler that is typically used during automated peritoneal dialysis.

Examples of a cycler are disclosed in U.S. patent applications: “Peritoneal Dialysis Systems and Methods Employing a Liquid Distribution and Pumping Cassette That Emulates Gravity Flow,” filed Mar. 3, 1993, Ser. No. 08/027,328; “Peritoneal Dialysis Systems and Methods Employing a Liquid Distribution and Pump Cassette with Self-Contained Air Isolation and Venting,” filed Mar. 3, 1993, Ser. No. 08/027,484; “Liquid Pumping Mechanisms for Peritoneal Dialysis Systems Employing Fluid Pressure,” filed Mar. 3, 1993, Ser. No. 08/027,485; “Peritoneal Dialysis Systems and Methods Employing Pneumatic Pressure and Temperature-Corrected Liquid Volume Measurements,” filed on Mar. 3, 1993, Ser. No. 08/026,458; “Improved User Interface for Automated Peritoneal Dialysis Systems,” filed Mar. 3, 1993, Ser. No. 08/025,531; “Improved User Interface for Automated Peritoneal Dialysis Systems,” filed Mar. 3, 1993, Ser. No. 08/025,547; and “Peritoneal Dialysis Cycler,” filed Mar. 3, 1993, Ser. No. 08/006,426, the disclosures of all of which are incorporated herein by reference. It should be appreciated that the cycler of the present invention can include any suitable modification of known cyclers in the art examples of which are described above.

As illustrated in FIGS. 3A and 3B, the pumping cassette 76 of the present invention includes the housing 78 that can enclose a number of fluid lines or pathways coupled to a respective port through which dialysis solution can flow during therapy. The cassette pumping mechanism 80 is coupled to the fluid lines 82 thereby causing the dialysis solution to flow into and out of the ports 84 and through the fluid lines 82. It should be appreciated that the pumping cassette 76 can be used during any suitable dialysis therapy, preferably during peritoneal dialysis including automated peritoneal dialysis and continuous flow peritoneal dialysis.

The pumping cassette 76 can include any number of suitable operational constituents in addition to the pumping mechanism 80. In an embodiment, the biosensor 86 of the present invention is integrated within the pumping cassette 76 as schematically shown in FIGS. 3A and 3B. The pumping cassette 76 includes a port 88 through which dialysis solution can flow into the biosensor 86. The biosensor 86 includes a fluid circuit 90 coupled to the port 88 and coupled to another port 84 via a fluid pathway such that fluid can flow out of the biosensor for disposal, reuse or the like. The fluid flow through the biosensor 86 can be regulated by valves 92 positioned at an in flow and out flow region of the biosensor 86.

The fluid circuit 90 can include any suitable design. As shown in FIG. 3B, the fluid circuit includes three monitoring pathways hydraulically coupled to the in flow port and the out flow port via three separate fluid lines. Each pathway can be used to monitor a separate component of the dialysis solution. The first pathway 94 and third pathway 96 use a reactive element 98 and a sensing element 100 in hydraulic connection to the fluid lines. In this regard, the component of the dialysate first reacts with the reactive element, such as an enzyme impregnated within a membrane, to produce a reaction product. The sensing element is optically sensitive to the level of reaction product such that the concentration of the component in the dialysis solution can be determined based on the optical measurement, preferably a calorimetric measurement as discussed above.

The second monitoring pathway 102 includes a single sensing element 104. This can be used to monitor infection in the dialysis solution based on measuring white blood cell counts, neutrophil counts or the like as discussed above.

It should be appreciated that the monitoring capabilities of the biosensor can be varied as desired during dialysis therapy. The reactive and/or sensing elements of the biosensor can be readily made as modules during manufacture. The cassette with specific sensing modules can have a designated barcode to be read by the optical reader in the dialysis instrument. This provides the user with the ability to select different modules depending on the application.

In this regard, the biosensor of the present invention can be readily adapted to provide a variety of different and suitable monitoring protocols during dialysis therapy. An illustrative example of the monitoring capabilities of the biosensor used during dialysis therapy in accordance with an embodiment of the present invention is described in detail below.

### BIOSENSOR MONITORING EXAMPLE

The following example demonstrates how the biosensor of the present invention can be used to monitor both solute removal levels and infection in the dialysis solution during therapy. Monitoring trends can then be utilized to prevent and/or treat infection, calculate solute clearance levels and/or the like. The trends can be assessed and used to make adjustments in the therapy and/or the like. The monitoring protocol of this example is applied to a peritoneal dialysis therapy that includes four nocturnal exchanges at 2 liters for each exchange with a dwell time of about 120 minutes between each exchange. In addition, the therapy includes one 2 liters daytime exchange with a dwell time of about fourteen hours before it is exchanged.

During the day, the pumping cassette is adapted to fill the peritoneum of the patient with the dialysate solution. The dialysate then dwells within the patient as discussed above after which it is drained from the patient via the pumping cassette. During drainage of the dialysis solution, the spent dialysate is fed into the biosensor integrated within the pumping cassette to monitor various parameters associated with dialysis therapy.

The first monitoring pathway is utilized to monitor a solute removed from the patient, such as urea, uric acid or creatinine. The desired component is first reacted with an enzyme specific to the desired component, e.g., urease for urea, as previously discussed. The concentration of this component can then be measured based on the colorimetric response of the sensing element within the first monitoring pathway. The third monitoring pathway is used to measure the concentration of a solute in the dialysis solution that is different than the solute monitored in the first monitoring pathway. The second monitoring pathway is used to monitor the presence of infection in the dialysis solution. For example, the white blood cell count, the neutrophil count and/or the like can be measured to assess the presence of infection.

The measurement data can be used to evaluate a number of different parameters. The calorimetric measurement data can be used to calculate the plasma concentration of the waste dialysis solution (“Cₚₗₐₛₘₐ”). The infection data can be evaluated and compared against standard levels. If the measured level of infection does not meet standard levels, this can trigger an alarm (not shown) or the like to alert the user such that responsive measures can be taken. In addition, a metering device or the like (not shown) can be utilized to measure the volume of dialysate drained from the patient (“V_{drain}”).

After draining the day-time dialysate fill, the pumping cassette then fills the patient with a fresh source of dialysate which dwells within the patient as described above. After the dwell period, the spent dialysate is drained, and the patient is filled with fresh dialysate. The series of fills and exchanges occurs four separate times as discussed above. After the last fill and dwell period, a last bag of fresh dialysate is supplied to the patient which dwells within the patient until it is exchanged with the daytime source of fresh dialysate. The daily fill, dwell and exchange of dialysis solution can be repeated each day throughout the week.

During the drainage or exchange of the dialysis solution associated with the first, second, third and fourth fill and dwell periods, the spent dialysis solution is supplied to the biosensor for monitoring purposes. The second monitoring pathway can be utilized to monitor the level of infection as discussed above. The volume of the drained dialysate can be measured. Further, the concentration of a solute or solutes removed from the patient can be calorimetrically measured using the first monitoring pathway and/or the third monitoring pathway as discussed above.

The measurement data can then be utilized to calculate clearance levels of desirable monitored solutes which have been removed from the patient during therapy. For example, clearance levels associated with urea, creatinine, uric acid and/or the like can be calculated on a daily basis and a weekly basis as indicated below.

Daily Clearance (“K_{day}”) calculations:

K_{day}=V_{drain}+[C1V1+C2V2+C3V3+C4V4]/Cₚₗₐₛₘₐ

where V_{drain} is the drained dialysate volume of the day dwell; C1, V1 are the solute concentration and drained dialysate volume at the end of the first exchange; C2, V2 are the solute concentration and drained dialysate volume at the end of the second exchange; C3, V3 are the solute concentration and drained dialysate volume at the end of the third exchange; and C4, V4 are the solute concentration and drained dialysate volume at the end of the fourth exchange. Cₚₗₐₛₘₐ represents the solute concentration in the plasma sample. In most cases, Cₚₗₐₛₘₐ can be approximated by the C_{drain} of the day dwell. It should be appreciated that the concentration and volume data can be defined by any suitable dimensions, such as moles per liter for the concentration data and liters for the volume data. The clearance values are in volume units Further, weekly clearance calculations can be determined for any suitable solute, such as urea, creatinine and/or the like.

Weekly Clearance (“Kt/V”) calculations:

Kt/V=(K_{day}×7)/distribution volume (estimated from anthropometric equation)

The amount of ultrafiltrate added to the dialysis solution during therapy can be calculated as follows:

V_{ultrafiltrate}=V_{drain}+V1+V2+V3+V4−(volume infused)

With the daily and weekly monitoring of clearance, amount of ultrafiltrate, infection and/or other suitable parameters, long-term trend analysis of dialysis therapy can be performed. For example, current solute removal levels, such as urea, can be compared to prior removal levels to assess the effectiveness of therapy over time. Adjustments in therapy can then be made to enhance therapy based on the results of the comparative analysis.

In an embodiment, the dialysis system and biosensor can communicate with one another such that the measured amounts of analytes removed during dialysis can be utilized to monitor total solute removals, clearances, like parameters or combinations thereof during dialysis therapy. This communication can be either hardwired (e.g., electrical communication cable), wireless communication, a pneumatic interface, the like or combinations thereof.

In this regard, data from the biosensor array (e.g., measurable amounts of analytes removed during dialysis) can be inputted into the dialysis system and vice versa for further processing. For example, analyte removal data can be utilized to determine when clearance levels in dialysis have been met. This event can then trigger a number of different responsive actions to be carried out by the dialysis machine and/or the biosensor, such as the activation a suitable alarm (e.g., audio, visual, the like or combinations thereof) to indicate when clearance levels have been met, thus signaling an endpoint of dialysis therapy. The detection of infection can initiate responsive actions, such as administering antibiotics to the patient or other medical treatments for infection. The progress of treatment can then be monitored.

Analyte Measurements

Applicants have conducted a number of experiments to demonstrate the effectiveness of the biosensor of the present invention. In general, Applicants have found that the complete conversion (e.g., effectively 100%) of analyte to measurable reaction products can be carried out with a sufficient enzyme quantity and/or a controlled flow rate. With respect to the optical detection of the measurable reaction products, Applicants have conducted a series of tests to demonstrate the accuracy and/or sensitivity of the sensing membrane layer, particularly with respect to the detection of ammonium, ammonia and pH.

With respect to the detection of ammonia and ammonium, Applicants prepared a number of test samples that included varying amounts of ammonia and ammonium derived from the enzymatic conversion of urea by urease and creatinine by creatinine dieiminase. The test results showed that the ammonium and ammonia sensing membrane was reliably able to optically measure the amount of ammonium and ammonia ranging from about 0.2 ppm to about 800 ppm which corresponds to urea and/or creatinine concentrations ranging from about 0.2 mg/dL to about 133 mg/dL.

This determination was based on a correlation (R2=0.99) of the optical measurements of ammonia and ammonium and calculations thereof associated with the sensing membrane of the present invention versus the ammonia and ammonium contents from about 0.1 ppm to about 800 ppm of the test solutions measured by a reference device (COBAS MIRA by ROCHE DIAGNOSTICS).

With respect to pH measurements, Applicants prepared a number of test samples from a dialysate test solution spiked with varying amounts of a bicarbonate solution. The pH of the test samples ranged from about 5.0 to about 8.17 depending on the amount of bicarbonate solution. The pH sensitive membranes tested were provided by OCEAN OPTICS, INC.

The test results showed that the pH sensitive membranes of the biosensor array of the present invention were optically sensitive to a change in pH level with error of pH 0.05.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. An apparatus for providing on-line monitoring of multiple parameters associated with a dialysate solution used during dialysis therapy, the apparatus comprising:
a housing enclosing a fluid circuit in hydraulic connection with the dialysate solution wherein the dialysate solution contains a plurality of constituents;
a plurality of reactive elements hydraulically coupled to the fluid circuit within the housing wherein the reactive elements are capable of reacting with at least a portion of the constituents of the dialysate solution to yield a reaction product; and
a plurality of sensing elements hydraulically coupled to the fluid circuit within the housing wherein the sensing elements are optically responsive to at least a portion of the constituents of the dialysate solution including the reaction product associated with the constituents such that an amount of the constituents in the dialysate solution is measurable.

2. The apparatus of claim 1 wherein the reactive elements each include an enzyme selected from the group consisting of urease, creatinine deiminase, uricase, leukocyte esterase and combinations thereof allowing the reactive elements to enzymatically react with at least a portion of the constituents to form the reaction product.

3. The apparatus of claim 1 wherein the apparatus includes a plurality of carrier media capable of supporting the reactive elements and the sensing elements wherein the carrier media are selected from the group consisting of a membrane, a substrate made from a fibrous material including paper and combinations thereof.

4. The apparatus of claim 1 wherein the constituents are selected from the group consisting of urea, creatinine, uric acid, glucose, phosphates, sodium, potassium, calcium, magnesium, pH, white blood cell count, total protein concentration, bacteria, bacterial wall components including endotoxin, lipid A, peptidoglycan, lipid A, muramyl peptide and P glycan levels, mediators of infection and combinations thereof.

5. The apparatus of claim 1 wherein the constituents of the dialysate solution comprise one or more analytes representative of solutes removed from a patient during dialysis therapy and one or more constituents representative of infection in the dialysate solution.

6. The apparatus of claim 5 wherein the analytes are measured to provide on-line monitoring of solute removal levels.

7. The apparatus of claim 1 wherein the apparatus is in wireless communication with a dialysis system to provide on-line monitoring during dialysis therapy.

8. The apparatus of claim 7 wherein the apparatus comprises a stand alone device in fluid connection with the dialysis system to provide on-line monitoring during dialysis therapy.

9. The apparatus of claim 1 wherein the housing has a configuration of a mini-cassette.

10. The apparatus of claim 1 wherein the sensing elements are connected to an opto-electrical circuit that is coupled to a microprocessor for converting the optical response into a concentration value associated with the constituents in the dialysate solution.

11. The apparatus of claim 1 wherein the apparatus is integrated within a pumping cassette used during dialysis therapy.

12. A pumping cassette comprising:
a housing enclosing a plurality of fluid lines through which a dialysate solution containing a plurality of constituents can flow during dialysis therapy;
a pumping mechanism coupled to one or more of the fluid lines wherein the pumping mechanism is capable of causing the dialysate solution to flow; and
a biosensor coupled to one or more of the fluid lines that is capable of providing on-line monitoring of a plurality of parameters associated with the dialysate solution, the biosensor includes a biosensor housing enclosing a biosensor fluid circuit in hydraulic connection with the dialysate solution, one or more reactive elements hydraulically coupled to the biosensor fluid circuit within the biosensor housing wherein the reactive elements are capable of reacting with at least a portion of the constituents of the dialysate solution to yield a reaction product, and one or more sensing elements hydraulically coupled to the biosensor fluid circuit within the biosensor housing wherein the sensing elements are optically responsive to at least a portion of the constituents of the dialysate solution including the reaction product associated with the constituents such that an amount of the constituents of the dialysate solution is measurable.

13. The pumping mechanism of claim 12 wherein the reactive elements of the biosensor each include an enzyme selected from the group consisting of urease, creatinine deiminase, uricase, leukocyte esterase and combinations thereof such that the reactive elements are capable of enzymatically reacting with at least a portion of the constituents to form the reaction product.

14. The pumping mechanism of claim 12 wherein the apparatus includes a plurality of carrier media capable of supporting the reactive elements and the sensing elements wherein the carrier media are selected from the group consisting of a membrane, a substrate made from a fibrous material including paper and combinations thereof.

15. The pumping mechanism of claim 12 wherein the constituents of the dialysate solution comprise one or more analytes representative of solutes removed from a patient during dialysis therapy and one or more constituents representative of infection in the dialysate solution.

16. The pumping mechanism of claim 15 wherein the constituents are selected from the group consisting of urea, creatinine, uric acid, glucose, phosphates, sodium, potassium, calcium, magnesium, pH, white blood cell count, total protein concentration, bacteria, bacterial wall components including endotoxin, lipid A, peptidoglycan, muramyl peptide and β glycan levels, mediators of infection and combinations thereof.

17. The pumping mechanism of claim 12 wherein the analytes are measured to provide on-line monitoring of solute removal levels.

18. A method of providing on-line monitoring of a dialysate solution used during dialysis therapy, the method comprising the steps of:
providing a biosensor defining a fluid circuit including a plurality of reactive elements and sensing elements that can be placed in fluid communication with a fluid channel;
supplying the dialysate solution to the fluid circuit via the fluid channel wherein the dialysate solution includes a plurality of constituents;
reacting at least a portion of the constituents with at least a portion of the reactive elements to produce a reaction product associated with the constituents;
optically measuring at least a portion of the constituents including the reaction product via the sensing elements; and
determining a concentration of the constituents in the dialysate solution using the optical measurement.

19. The method of claim 18 wherein the reactive elements and the sensing elements are supported on a plurality of carrier members selected from the group consisting of a membrane, a substrate made from a fibrous material including paper and combinations thereof.

20. The method of claim 18 wherein the constituents of the dialysate solution enzymatically react with an enzyme of the reactive elements selected from the group consisting of urease, creatinine deiminase, uricase, leukocyte esterase and combinations thereof.

21. The method of claim 18 wherein the constituents of the dialysate solution comprise one or more analytes representative of solutes removed from a patient during dialysis therapy and one or more measurable constituents indicative of infection.

22. The method of claim 21 wherein the analytes and the constituents indicative of infection are calorimetrically measured to provide on-line monitoring during dialysis therapy.

23. The method of claim 22 further comprising the step of calculating clearance values based on the analyte measurements and concentration of the analytes measured at certain time intervals prior to removal from the patient during dialysis therapy.

24. The method of claim 21 wherein the constituents are selected from the group consisting of urea, creatinine, uric acid, glucose, phosphates, sodium, potassium, calcium, magnesium, pH, white blood cell count, total protein concentration, bacteria, bacterial wall components including endotoxin, lipid A, peptidoglycan, muramyl peptide and β glycan levels, mediators of infection and combinations thereof.

25. The method of claim 24 wherein a presence of infection due to peritonitis is monitored based on calorimetrically measuring the constituents selected from the group consisting of white blood cell count, total protein concentration, bacteria, bacterial wall components including endotoxin, lipid A, peptidoglycan, muramyl peptide and β glycan levels, mediators of infection and combinations thereof.

26. A method for providing dialysis therapy comprising the steps of:
using a dialysate solution to remove one or more solutes from blood of a patient;
supplying the dialysate solution to a biosensor array including a housing that encloses a fluid circuit in fluid communication with a plurality of reactive elements and sensing elements wherein the dialysate contains a plurality of constituents including the solutes removed from the patient;
reacting at least a portion of the constituents of the dialysate solution with the reactive elements to produce a reaction product associated with the constituents;
optically sensing at least a portion of the constituents including the reaction product with the sensing elements; and
determining an amount of the constituents in the dialysate solution using data generated from the optically sensing step.

27. The method of claim 26 wherein the reactive elements and the sensing elements are supported on a plurality of carrier media selected from the group consisting of a membrane, a substrate made from a fibrous material including paper and combinations thereof.

28. The method of claim 26 wherein the constituents of the dialysate solution enzymatically react with an enzyme of the reactive elements selected from the group consisting of urease, creatinine deiminase, uricase, leukocyte esterase and combinations thereof.

29. The method of claim 26 wherein the constituents of the dialysate solution comprise one or more analytes representative of the solutes removed from the patient during dialysis therapy and one or more constituents indicative of infection in the dialysate solution.

30. The method of claim 29 wherein the constituents are selected from the group consisting of urea, creatinine, uric acid, glucose, phosphates, sodium, potassium, calcium, magnesium, pH, white blood cell count, total protein concentration, bacteria, bacterial wall components including endotoxin, lipid A, peptidoglycan, muramyl peptide and β glycan levels, mediators of infection and combinations thereof.

31. The method of claim 29, further comprising the step of providing online monitoring of an infection due to peritonitis based on measuring the constituents indicative of infection.

32. The method of claim 31 further comprising the steps of treating the infection and monitoring progress of treatment of the infection based on measuring the constituents indicative of the infection.

33. The method of claim 29 wherein the analytes are measured to provide on-line monitoring of solute removal levels during dialysis therapy.

34. The method of claim 33 further comprising the step of calculating clearance values based on the on-line monitoring of solute removal levels and a concentration of the solutes measured at certain time intervals prior to removal from the patient during dialysis therapy.
